SEQUENCE LISTING (14 sequences)

SEQ ID NO: 1 - DNA, 576 bp, synthetic construct
  source 1..576: mol_type = other DNA
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 2 - protein, 191 aa, synthetic construct [sequence omitted]

SEQ ID NO: 3 - DNA, 576 bp, synthetic construct
  source 1..576: mol_type = other DNA
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 4 - protein, 191 aa, synthetic construct
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQTYSKFDTNSHNDDALLKNYGLLYCFRKDMDKVETFLRIVQCRSVEGSCGF

SEQ ID NO: 5 - DNA, 591 bp, synthetic construct
  source 1..591: mol_type = other DNA
ggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 6 - protein, 196 aa, synthetic construct
GGSSGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQCYSKFDTNSHNDDALLKNYGLLYCFRKDMDKVETFLRIVQCRSVEGSCGF

SEQ ID NO: 7 - DNA, 579 bp, synthetic construct
  source 1..579: mol_type = other DNA
atgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 8 - protein, 192 aa, synthetic construct
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQCYSKFDTNSHNDDALLKNYGLLYCFRKDMDKVETFLRIVQCRSVEGSCGF

SEQ ID NO: 9 - DNA, 576 bp, synthetic construct
  source 1..576: mol_type = other DNA
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 10 - protein, 191 aa, synthetic construct
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQCYSKFDTNSHNDDALLKNYGLLYCFRKDMDKVETFLRIVQCRSVEGSCGF

SEQ ID NO: 11 - DNA, 579 bp, synthetic construct
  source 1..579: mol_type = other DNA
atgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcatgcaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 12 - protein, 192 aa, synthetic construct
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQCYSKFDTNSCNDDALLKNYGLLYCFRKDMDKVETFLRIVQCRSVEGSCGF

SEQ ID NO: 13 - protein, 5 aa, synthetic construct
GGSSG

SEQ ID NO: 14 - protein, 6 aa, synthetic construct
GGSSGF

(19) United States
(12) Patent Application Publication
(10) Pub. No.: US 2025/0099550 A1
(43) Pub. Date: Mar. 27, 2025

(54) METHODS FOR TREATING ALZHEIMERS DISEASE AND RELATED DEMENTIAS AND AGING USING PEGYLATED GROWTH HORMONE RECEPTOR ANTAGONISTS

(71) Applicant: Molecular Technologies Laboratories LLC, Galena, OH (US)

(72) Inventors: Richard S. Brody, Galena, OH (US); Thomas J. Zupancic, Galena, OH (US); Uday Sandbhor, Galena, OH (US); John J. Kopchick, Galena, OH (US); Reetobrata Basu, Galena, OH (US); Sofiya Milman, Galena, OH (US); Derek Huffman, Galena, OH (US)

(21) Appl. No.: 18/893,544

(22) Filed: Sep. 23, 2024

Related U.S. Application Data
(60) Provisional application No. 63/539,874, filed on Sep. 22, 2023.

(51) Int. Cl. A61K 38/27; A61K 47/60; A61P 25/28

(52) U.S. Cl. CPC A61K 38/27; A61K 47/60; A61P 25/28

(57) ABSTRACT

A method for treating Alzheimer's disease and related dementias and for slowing or delaying aging and prolonging life, comprising reducing circulating IGF-1 levels by administering to the patient an effective amount of a human growth hormone receptor antagonist, comprising human growth hormone receptor antagonist G120K, wherein a single amino acid of the human growth hormone receptor antagonist G120K has been mutated to cysteine, and wherein the single amino acid mutated to cysteine is T142; and a polydispersed 40 kDa branched polyethylene glycol molecule conjugated to the substituted cysteine in the human growth hormone receptor antagonist G120K mutant, wherein reducing circulating IGF-1 levels reduces the effects of Alzheimer's disease and related dementias and slows or delays aging and prolongs life.

### CROSS-REFERENCE TO RELATED APPLICATIONS

This patent application claims the benefit of U.S. Provisional Patent Application Ser. No. 63/539,874 filed on Sep. 22, 2023 and entitled “Methods for Treating Alzheimer's Disease and Related Dementias Using Pegylated Growth Hormone Receptor Antagonists” the disclosure of which is hereby incorporated by reference herein in its entirety and made part of the present U.S. utility patent application for all purposes.

### REFERENCE TO A SEQUENCE LISTING

A sequence listing in computer readable form (CRF) is on file. The sequence listing is in an XML file entitled 44989-0018-U1.xml created on Sep. 23, 2024 and is 17 KB in size. The sequence listing is incorporated by reference as if fully recited herein.

### BACKGROUND

The disclosed technology relates in general to the therapeutic use of pegylated growth hormone antagonists, and more specifically to methods for treating Alzheimer's disease and related dementias using a specific pegylated growth hormone receptor antagonist.

Despite major efforts by the research community, effective therapies for Alzheimer's disease (AD) and related dementias (ADRDs) remain elusive. Aging is the major risk factor for ADO and ADRD, which indicates that innovative approaches and therapies based on discoveries in populations that delay aging and AD should be prioritized. Notably, centenarians are resilient to dementia despite advanced chronological age [1,2] and are enriched with genetic variants that reduce somatotropic signaling [3,4]. Indeed, there is compelling evidence that reduction in growth hormone/insulin-like growth factor 1 (GH/IGF-1) signaling delays aging, resulting in longer lifespan and dementia-free survival in model organisms [5-9] and humans [3,10-14]. Circulating insulin-like growth factor-1 (IGF-1) is a hormone that is produced in the liver and circulates in the blood, bound to binding proteins. IGF-1 functions as the major mediator of growth hormone (GH)-stimulated somatic growth, as well as a mediator of GH-independent anabolic responses in many cells and tissues. IGF-1 is a small peptide (molecular weight 7647) that circulates in serum bound to high affinity binding proteins. Additionally, lower circulating IGF-1 levels were associated with preserved cognition in people with exceptional longevity and predicted reduced risk of developing cognitive impairment in older adults [15]. Furthermore, a study of over 400,000 individuals from the UK Biobank revealed that highest quintile of serum IGF-1 levels at baseline was associated with greater than 20% increased hazard for all-cause dementia [16]. Elevations in GH/IGF-1 have similarly been linked to 30% excess risk of incident AD in patients with acromegaly, a disease of GH hypersecretion [17]. The pathogenesis of AD is noted to be multifactorial, while reduction in GH/IGF-1 signaling has pleiotropic effects, which may explain why attenuation in GH/IGF-1 may be effective in protecting from or delaying AD. Attenuated GH/IGF-1 signaling can reduce Aß plaque burden, oxidative stress, neuroinflammation, hyperphosphorylated tau, vascular dysfunction, and metabolic dysfunction, while also upregulating autophagy [18-20]. Thus, genetic attenuation of this pathway is often linked to delayed cognitive decline and enhanced learning ability and memory in multiple aged and AD rodent models [18,20,21].

### Attenuated GH/IGF-1 Signaling Delays Aging and Age-Related Cognitive Decline

Aging is a major risk factor for Alzheimer's disease (AD) and dementia. Thus, understanding the biological drivers that delay aging may lead to the identification of novel mechanisms that protect against AD. The growth hormone/insulin-like growth factor-1 (GH/IGF-1) signaling axis, is a conserved biological pathway that has emerged as one of the leading regulators of the aging process and lifespan. Attenuation of GH/IGF-1 signaling has been shown to reduce the incidence of many age-related diseases, including AD and dementia, and extend survival not only in invertebrate [7] and rodent models [5,6,8,9], but also in humans [3,10-12,14, 23]. In fact, centenarians, many of whom significantly delay and frequently avoid the onset of dementia despite the risk of advanced chronological age [1,2,24], display genotypes and phenotypes associated with decreased somatotropic signaling [3,10,12,13]. Females with exceptional longevity and lower serum IGF-1 levels have been shown not only to exhibit extended survival, but also were 50% less likely to develop cognitive impairment after age 95, compared to their contemporaries with higher IGF-1 levels [12,13]. Additionally, it has been demonstrated that older adults with higher baseline circulating IGF-1 levels had greater incidence of multidomain cognitive impairment [15]. This finding was subsequently confirmed in the UK Biobank cohort of over 400,000 individuals, where among older age groups the highest quintile of serum IGF-1 measured at baseline was associated with greater than 20% increased hazard for all-cause dementia 16. Thus, attenuated GH/IGF-1 signaling may play an important role in delayed aging and protection against AD and AD related dementias (ADRD).

### Regulation of GH/IGF-1 Axis

Growth hormone (GH) is secreted from the pituitary gland in pulses under the stimulation of GH releasing hormone (GHRH) and ghrelin that act by binding to their respective receptors. GH binding to the GH receptor (GHR) stimulates IGF-1 expression, which in turn inhibits GH and GHRH secretion in a negative-feedback loop. An additional negative regulator of GH secretion is somatostatin, produced in hypothalamic neurons. GH is a major regulator of circulating IGF-1 with the liver being the main source of peripheral IGF-1 production [25]. Hepatic-derived IGF-1 comprises about 75% of total circulating IGF-125 and crosses the blood brain barrier at the choroid plexus by way of the IGF-1 receptor (IGF-1R) and low-density lipoprotein receptor-related protein 2 transport protein into the cerebrospinal fluid [26]; thus, serum IGF-1 contributes a substantial portion to the total IGF-1 pool of the central nervous system. IGF-1 binds to six structurally related proteins, termed IGF-binding proteins (IGFBP-1 to 6), that sequester IGF-1 in high-affinity complexes. In circulation, most of the IGF-1 is bound to IGFBP-3 as part of a ternary complex involving a third protein, the acid-labile subunit (ALS) [27]. This process protects IGF-1 and IGFBPs from rapid degradation and keeps the circulating concentrations of biologically active, unbound IGF-1 tightly regulated [28,29]. Determination of 24-hour GH production requires repeated sampling over the course of the day given its pulsatile secretion and short half-life of only 20 minutes. The IGF-1 complex, on the other hand, has a half-life of 16 hours and can serve as a surrogate “read-out” of GH levels. IGFBP-3 is also induced by GH and can be used as another read-out of GH action. IGF-1 initiates a complex intracellular signaling cascade by binding to the high-affinity IGF-IR and insulin receptor (INSR) on the cell surface and activating the phosphorylation of insulin receptor substrate (IRS) molecules and the phosphoinositide 3-kinase-protein kinase B (PI3K-Akt) signaling cascade that regulates downstream mechanistic target of rapamycin (mTOR) activity30. mTOR interacts with other proteins to form mTOR complex 1 (mTORC1) that inactivates transcription factor EB (TFEB). mTORC1 inhibition, on the other hand, that occurs in the setting of decreased GH/IGF-1 signaling results in translocation of TFEB to the nucleus and stimulates transcription of autophagy genes, thereby activating autophagy that has been implicated in cognitive resilience [32].

### GH/IGF-1 Axis and the Aging Brain

IGF-1 levels vary considerably throughout the lifespan, peaking in the 2nd decade of life, an age that coincides with rapid cell proliferation and linear growth, then dropping precipitously until the 6th decade and stabilizing thereafter [33], but with substantial inter-individual variation (see FIG. 1). The physiological decline in IGF-1 with aging is attributed to diminished GH pulse amplitude and frequency resulting in steady secretion of low levels of GH [34,35]. Although IGF-1 is required for normal brain development [36-39] and is likely beneficial for repair after an acute neuronal injury, such as traumatic brain injury (TBI) [40], its role in the aging brain is far less clear. In fact, acromegaly, a disorder associated with excess GH secretion is associated with a nearly 30% increased hazard of incident all-cause dementia and AD [17]. Additionally, most clinical trials of GH/IGF-1 replacement showed no beneficial effect on cognition or progression of AD [41,42]. On the other hand, individuals with GHR deletion (Laron Syndrome) are resistant to GH action and exhibit enhanced cognitive performance and greater task-related activation in frontal, parietal, and hippocampal regions of the brain, reflective of younger brain structure and function [43]. Moreover, recent evidence indicates that lower IGF-1 levels in older adults are also associated with protection from other known AD risk factors, including incident vascular disease and diabetes, as well as all-cause mortality [16]. Thus, most evidence suggests that reduced GH/IGF-1 signaling is likely beneficial for aged individuals who are at risk for progressive neurodegenerative disorders, such as AD. Attenuation of GH/IGF-1 leads to up-regulation of cell-maintenance through enhanced autophagy and reduction in AB plaque formation, tau phosphorylation, neuroinflammation, oxidative stress, metabolic alterations and vascular dysfunction [18-20].

### Pleiotropic Effects of Attenuated GH/IGF-1 Signaling in the AD Brain

It is well recognized that the etiology of AD is multifactorial, involving Aβ plaque formation, tau hyperphosphorylation, neuroinflammation, oxidative stress, metabolic alterations, dysregulated autophagy and vascular dysfunction. Mounting evidence from in vivo and in vitro studies demonstrates that attenuation of GH/IGF-1 signaling has pleotropic effects on the aging brain, cognitive function, and progression of AD, impacting all of the aforementioned pathogenic processes. Mice that have very low serum IGF-1 levels due to lack of GH or GHR exhibit increased longevity, as well as improved learning and memory at old age [8,9]. Similarly, GHR antagonist transgenic mice (GHA mice) demonstrate improved spatial learning and memory compared to wild type animals at 12 months [21]. Rodent AD models that express human mutant amyloid precursor protein (APP) and presenilin-1 (PS-1) display reductions in Aβ plaque deposition, neuroinflammation, and neurodegeneration, as well as improved spatial memory and delayed death when genetically modified to co-express mutations that reduce GH/IGF-1 signaling [18-20,44]. Treatment of 5XFAD mice with subcutaneous GHRH antagonist resulted in reduction of Aβ and tau and enhancement in anti-oxidation [45]. Also, knocking out (KO) IGF-1R in neurons resulted in better autophagy and clearance of Aβ plaques [19], whereas prolonged exposure to IGF-1 resulted in decreased autophagy in human fibroblasts [46]. However, there was no benefit to reducing somatotropic signaling in an advanced model of AD47, suggesting that there may be a limited window for intervention. A number of studies in human and rodent AD brains found resistance to IGF-1 at the point of IGF-1R/IRS-2 signaling [48-50]. Whether brain resistance to IGF-1 is a pathologic feature or a protective adaption remains uncertain. However, a recent genome-wide microarray analysis that compared neurons in early-stage AD with IGF-1R KO neurons, which are protected from AD, demonstrated very similar transcriptomic signatures, suggesting that IGF-1 resistance in AD neurons may be an early adaptation intended to protect neurons from further damage [47]. Thus, based on animal and in vitro models, diminished GH/IGF-1 signaling appears to protect mice from age- and AD-related neurodegeneration.

### SUMMARY

The following provides a summary of certain example implementations of the disclosed technology. This summary is not an extensive overview and is not intended to identify key or critical aspects or elements of the disclosed technology or to delineate its scope. However, it is to be understood that the use of indefinite articles in the language used to describe and claim the disclosed technology is not intended in any way to limit the described technology. Rather the use of “a” or “an” should be interpreted to mean “at least one” or “one or more”.

One implementation of the disclosed technology provides a method for treating diseases or conditions responsive to human growth hormone receptor antagonists, comprising administering to the patient an effective amount of a human growth hormone receptor antagonist, comprising human growth hormone receptor antagonist G120K, wherein a single amino acid of the human growth hormone receptor antagonist G120K has been mutated to cysteine, and wherein the single amino acid mutated to cysteine is T142; and a polydispersed 40 kDa branched polyethylene glycol molecule conjugated to the substituted cysteine in the human growth hormone receptor antagonist G120K mutant. In certain implementations, the diseases or conditions include Alzheimer's disease and related dementias. In other implementations, the diseases or conditions include aging. In certain implementations, administering the human growth hormone receptor antagonist reduces circulating IGF-1 levels. In certain implementations, the human growth hormone receptor antagonist is encoded by a DNA molecule having the sequence consisting of SEQ ID NO: 7, and the amino acid sequence consisting of SEQ ID NO: 8. In certain implementations, the human growth hormone receptor antagonist is encoded by a DNA molecule having the sequence consisting of SEQ ID NO: 9, and the amino acid sequence consisting of SEQ ID NO: 10. The disclosed human growth hormone receptor antagonist may be administered to mammal, including humans.

Another implementation of the disclosed technology provides a method for treating Alzheimer's disease and related dementias, comprising reducing circulating IGF-1 levels by administering to the patient an effective amount of a human growth hormone receptor antagonist, comprising human growth hormone receptor antagonist G120K, wherein a single amino acid of the human growth hormone receptor antagonist G120K has been mutated to cysteine, and wherein the single amino acid mutated to cysteine is T142; and a polydispersed 40 kDa branched polyethylene glycol molecule conjugated to the substituted cysteine in the human growth hormone receptor antagonist G120K mutant, wherein reducing circulating IGF-1 levels reduces the effects of Alzheimer's disease and related dementias. In certain implementations, the human growth hormone receptor antagonist is encoded by a DNA molecule having the sequence consisting of SEQ ID NO: 7, and the amino acid sequence consisting of SEQ ID NO: 8. In certain implementations, the human growth hormone receptor antagonist is encoded by a DNA molecule having the sequence consisting of SEQ ID NO: 9, and the amino acid sequence consisting of SEQ ID NO: 10. The disclosed human growth hormone receptor antagonist may be administered to mammal, including humans. The disclosed human growth hormone receptor antagonist may be administered to mammal, including humans.

Still another implementation of the disclosed technology provides a method for delaying aging and prolonging lifespan, comprising reducing circulating IGF-1 levels by administering to the patient an effective amount of a human growth hormone receptor antagonist, comprising human growth hormone receptor antagonist G120K, wherein a single amino acid of the human growth hormone receptor antagonist G120K has been mutated to cysteine, and wherein the single amino acid mutated to cysteine is T142; and a polydispersed 40 kDa branched polyethylene glycol molecule conjugated to the substituted cysteine in the human growth hormone receptor antagonist G120K mutant, wherein reducing circulating IGF-1 levels delays or slows aging and prolongs lifespan.

It should be appreciated that all combinations of the foregoing concepts and additional concepts discussed in greater detail below (provided such concepts are not mutually inconsistent) are contemplated as being part of the technology disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the descriptions provided herein are to be regarded as illustrative and not restrictive in nature.

### BRIEF DESCRIPTION OF THE DRAWINGS

The accompanying drawings, which are incorporated into and form a part of the specification, schematically illustrate one or more example implementations of the disclosed technology and together with the general description given above and detailed description given below, serve to explain the principles of the disclosed subject matter, and wherein:

FIG. 1 is a graph depicting inter-individual variation in serum IGF-1 levels stratified by age, wherein the large black dot represents a mean IGF-1 level for the age group, and wherein the smaller colored dots represent individual IGF-1 levels;

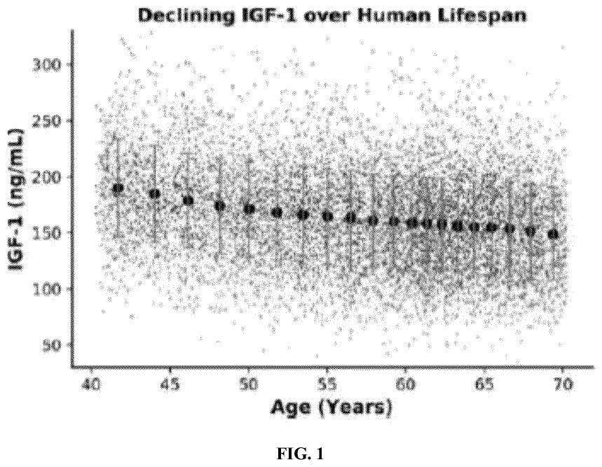

FIG. 2A and FIG. 2B provide data based on single intraperitoneal (IP) injections in an animal model demonstrating the efficacy of the disclosed compound (D₂) in substantially reducing circulating IGF-1 levels;

FIG. 2C and FIG. 2D provide data based on daily IP injections for 7 days in an animal model demonstrating the efficacy of the disclosed compound (D₂) in substantially reducing circulating IGF-1 levels;

FIG. 3A and FIG. 3B provide data based on single intraperitoneal (IP) injections in an animal model demonstrating the efficacy of an alternate compound (Compound G disclosed in U.S. Pat. No. 11,452,763; see also G₂ below) in substantially reducing circulating IGF-1 levels;

FIGS. 4A and 4B provide data based on daily IP injections for 7 days in an animal model demonstrating the efficacy of an alternate compound (Compound G disclosed in U.S. Pat. No. 11,452,763; see also G₂ below) in substantially reducing circulating IGF-1 levels;

FIG. 5A and FIG. 5B provide data based on single intraperitoneal (IP) injections in an animal model demonstrating the efficacy of Compound D₂ compared to Compound G in substantially reducing circulating IGF-1 levels; and

FIG. 6A and FIG. 6B provide data based on daily IP injections for 7 days in an animal model demonstrating the efficacy of Compound D₂ compared to Compound G in substantially reducing circulating IGF-1 levels.

### DETAILED DESCRIPTION

Example implementations are now described with reference to the Figures. Reference numerals are used throughout the detailed description to refer to the various elements and structures. Although the following detailed description contains many specifics for the purposes of illustration, a person of ordinary skill in the art will appreciate that many variations and alterations to the following details are within the scope of the disclosed technology. Accordingly, the following implementations are set forth without any loss of generality to, and without imposing limitations upon, the claimed subject matter.

The examples discussed herein are examples only and are provided to assist in the explanation of the apparatuses, devices, systems, and methods described herein. None of the features or components shown in the drawings or discussed below should be taken as required for any specific implementation of any of these the apparatuses, devices, systems or methods unless specifically designated as such. For ease of reading and clarity, certain components, modules, or methods may be described solely in connection with a specific Figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As disclosed in U.S. patent application Ser. No. 18/402,090, which is incorporated by reference herein, in its entirety, a novel hormone antagonist has been developed that binds to peripheral GHR and prevents expression and secretion of circulating IGF-1. This disclosed compound, referred to as Compound D (note three variants are disclosed: D₁, D₂, D₃) is significantly superior to the existing FDA-approved GHRA, pegvisomant, which has been effectively and safely used world-wide since 2003 for the treatment of acromegaly [22]. Compound D is a 191 amino acid protein conjugated to a 40 kDa PEG at a single site predicted not to participate in receptor binding. Compound D binds to the hGHR, blocking hGH binding (see U.S. Pat. No. 10,874,717) and subsequent GH induced intracellular signaling and downstream IGF-1 production. Unlike pegvisomant, Compound D is homogenous and demonstrates ˜20× greater affinity for the hGHR. Like pegvisomant, it has been shown in mice to have slow clearance rate, the same beneficial effects on insulin sensitivity, and the same excellent safety profile that will make it amenable to chronic administration in older adults to prevent or delay AD. With reference to the Figures, the data presented herein was generated with Compound D₂ and these results demonstrate the efficacy of the compound in substantially reducing circulating IGF-1 levels.

The disclosed compositions provide novel human growth hormone receptor antagonists that are useful in various therapeutic applications. TABLE 1, below, includes names and abbreviations for specific human growth hormone (hGH) antagonists disclosed herein. For reference purposes, SEQ ID NO: 1 provides the DNA sequence for human growth hormone WThGH and SEQ ID NO: 2 and provides the amino acid sequence for human growth hormone WThGH (mature form), referred to herein as hGH. Human growth hormone receptor antagonist hGH-G120K, referred to herein as G120K, is the parent receptor antagonist for the compositions of the disclosed technology, and for reference purposes, SEQ ID NO: 3 provides the DNA sequence for human growth hormone receptor antagonist G120K and SEQ ID NO: 4 provides the amino acid sequence for human growth hormone receptor antagonist G120K (mature form). A first disclosed pegylated growth hormone receptor antagonist having an increased serum half-life is GGSSG-hGH-G120K-T142C-GL2-400MA, referred to herein as Compound D₁, and for reference purposes, SEQ ID NO: 5 provides the DNA sequence for Compound D₁ and SEQ ID NO: 6 provides the amino acid sequence for Compound D₁. A second disclosed pegylated growth hormone receptor antagonist having an increased serum half-life is M-hGH-G120K-T142C-GL2-400MA, referred to herein as Compound D₂, and for reference purposes, SEQ ID NO: 7 provides the DNA sequence for Compound D₂ and SEQ ID NO: 8 provides the amino acid sequence for Compound D₂. A third disclosed pegylated growth hormone receptor antagonist having an increased serum half-life is hGH-G120K-T142C-GL2-400MA, referred to herein as Compound D₃, and for reference purposes, SEQ ID NO: 9 provides the DNA sequence for Compound D₃ and SEQ ID NO: 10 provides the amino acid sequence for Compound D₃. The single letter amino acid abbreviations used herein follow the IUPAC format.

TABLE 1
Designations for hGH and Mutant hGH Antagonists

| | | SED ID |
|---|---|---|
| Name | Abbreviation | NOS |
| hGH | hGH | 1-2 |
| G120K | hGH-G120K | 3-4 |
| Compound D₁ | GGSSG-hGH-G120K-T142C- | 5-6 |
| | GL2-400MA | |
| Compound D₂ | M-hGH-G120K-T142C-GL2-400MA | 7-8 |
| Compound D₃ | hGH-G120K-T142C-GL2-400MA | 9-10 |
| Compound G₂ | M-hGH-G120K-T142C-dPEGA- | 11-12 |
| | H151C-dPEGA | |

The preparation of Compound D₁ (GGSSG-hGH-G120K-T142C-GL2-400MA) is disclosed in U.S. Pat. No. 10,874,717. The preparation of Compound D₂ (M-hGH-G120K-T142C-GL2-400MA) is disclosed in U.S. patent application Ser. No. 18/402,090. The method disclosed is also relevant with regarding to preparing Compound D₃ (hGH-G120K-T142C-GL2-400MA; however, other methods for preparing Compound D₃ are possible and are utilized in alternate implementations.

With reference to TABLE 1, the three disclosed Compound D variants differ in the amino acids that are attached to the phenylalanine (F) that is the N-terminus of native hGH. Compound D₁ includes the sequence GGSSGF-on its N-terminus (see SEQ ID NO: 13), Compound D₂ includes an MF-on its N-terminus, and Compound D₃ begins with the native phenylalanine (F-). The affinities of D₁ and D₂ for the hGH receptor are essentially identical despite their differing by five N-terminal amino acids and are similar to the affinity of native hGH for the receptor. Variant D₃, which differs from D₂ by only a single amino acid will presumably exhibit a similar affinity for hGH receptor.

Compound D₂ contains a non-native an N-terminal methionine. Because the methionine is adjacent to a phenylalanine, the N-terminal amino acid of native growth hormone, it is not readily cleaved by the N-terminal methionine aminopeptidase of E. coli. However, the preparation of the antagonist without the methionine (D₃), having utility as a therapeutic, can be readily performed one of ordinary skill in the art. Compound D₃ can be produced without the N-terminal methionine by expression into the E. coli periplasm, expression into the cell medium, or expression by mammalian cells. Compound G₁ (GGSSG-hGH-G120K-H151C-T142C-dPEGA₂), which is disclosed in U.S. Pat. No. 10,874,717, can be made without the GGSSG terminus by published methods.

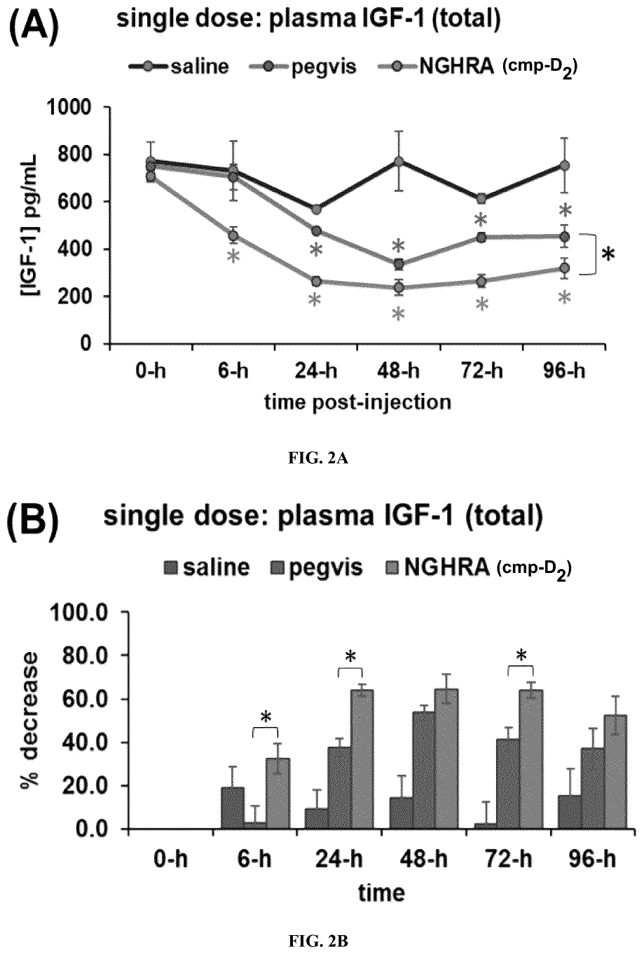

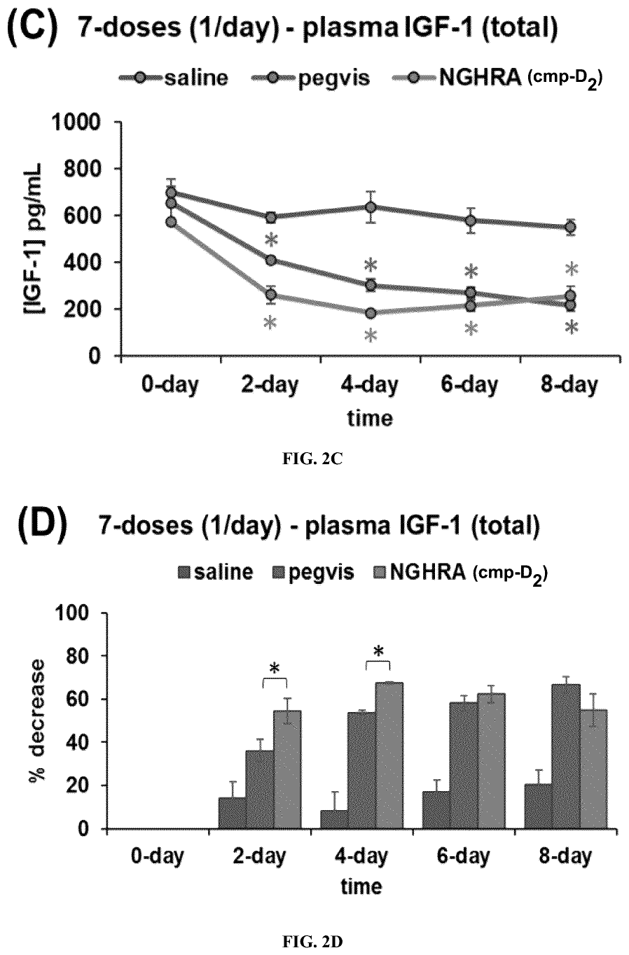

FIGS. 2A and 2B (*p<0.05) provide data for a single intraperitoneal (IP) injection. With regard to these Figures, 3-month-old C57BL/6J male mice (n=6, Jackson Lab) were treated with a single IP injection of 200 mg/kg of pegvisomant or Compound D₂ (NGHRA) or saline (control) and blood was collected to measure changes in plasma IGF-1 (total) at 0-h (pre-injection), and at 6-h, 24-h, 48-h, 72-h, and 96-h post-injection. At 6-h following a single 200 mg/kg dose of Compound D₂ or pegvisomant, circulating IGF-1 was lowered by 45% and 6%, respectively. Maximal IGF-1 suppression was observed at 48-h post-injection, with IGF-1 lowered by 66% and 55% with Compound D₂ and pegvisomant, respectively. Finally, at 96-h post-injection, IGF-1 levels remained suppressed by 55% and 40% in Compound D₂ and pegvisomant treated mice, respectively, indicating superior in vivo efficacy of Compound D₂ over that of pegvisomant.

FIGS. 2C and 2D, (*p<0.05) provide data for daily IP injections for 7 days. With regard to these Figures, 3-month-old C57BL/6J male mice (n=3, Jackson Lab) were also treated with seven, once daily, IP injections of 100 mg/kg of pegvisomant or Compound D₂ or saline (control) and blood was periodically collected to measure changes in plasma IGF-1 (total) at day-0 (pre-injection), and day-2, day-4, day-6, and day-8 post-injection. At this dose and administration frequency, the circulating IGF-1 levels initially decreased more rapidly and drastically with Cpd D₂ compared to pegvisomant; however, by day-8 (end-of-study), both Cpd D₂ and pegvisomant demonstrated similar suppression of total IGF-1 levels. These results indicate that the Cpd D₂ can achieve effective and sustained suppression of GH/IGF-1 signaling and can likely be administered at intervals less frequent than daily.

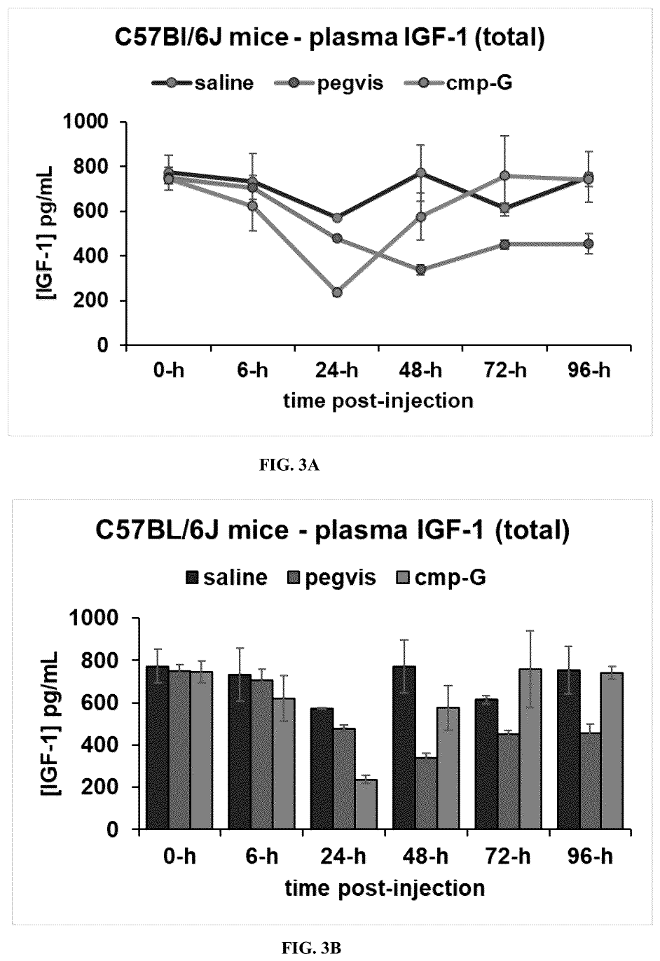

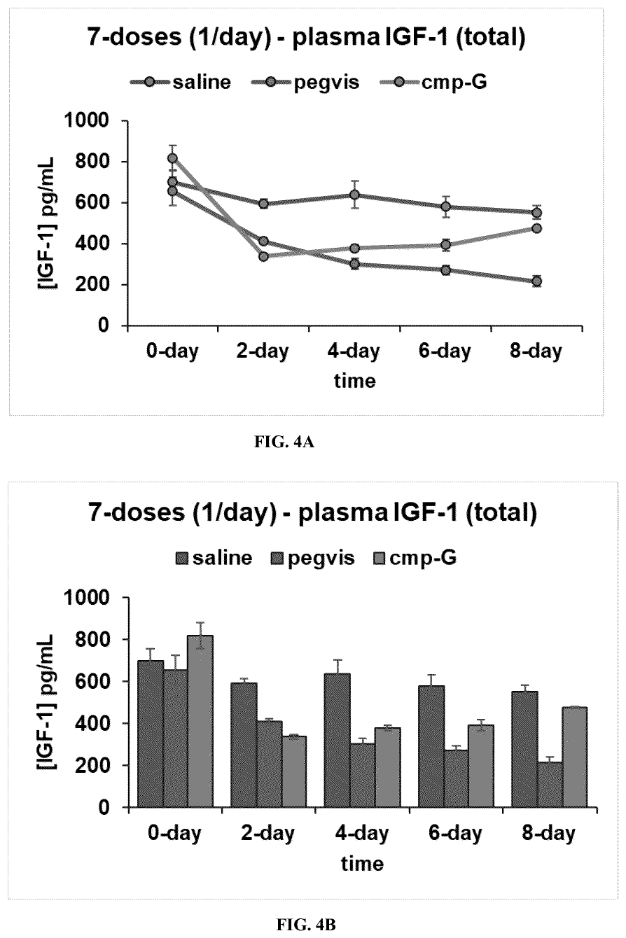

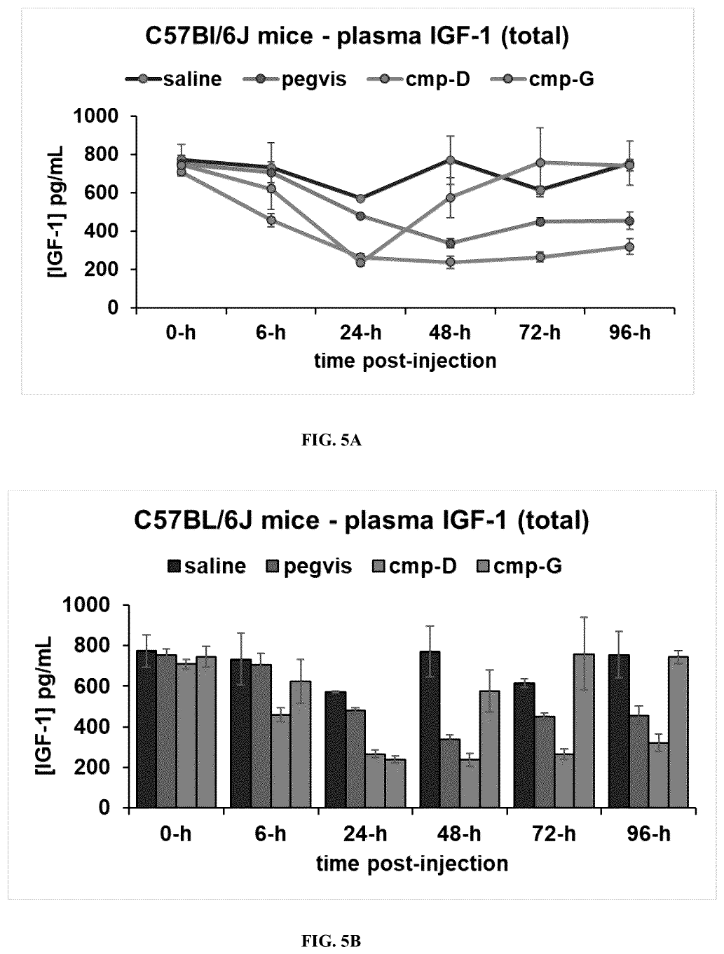

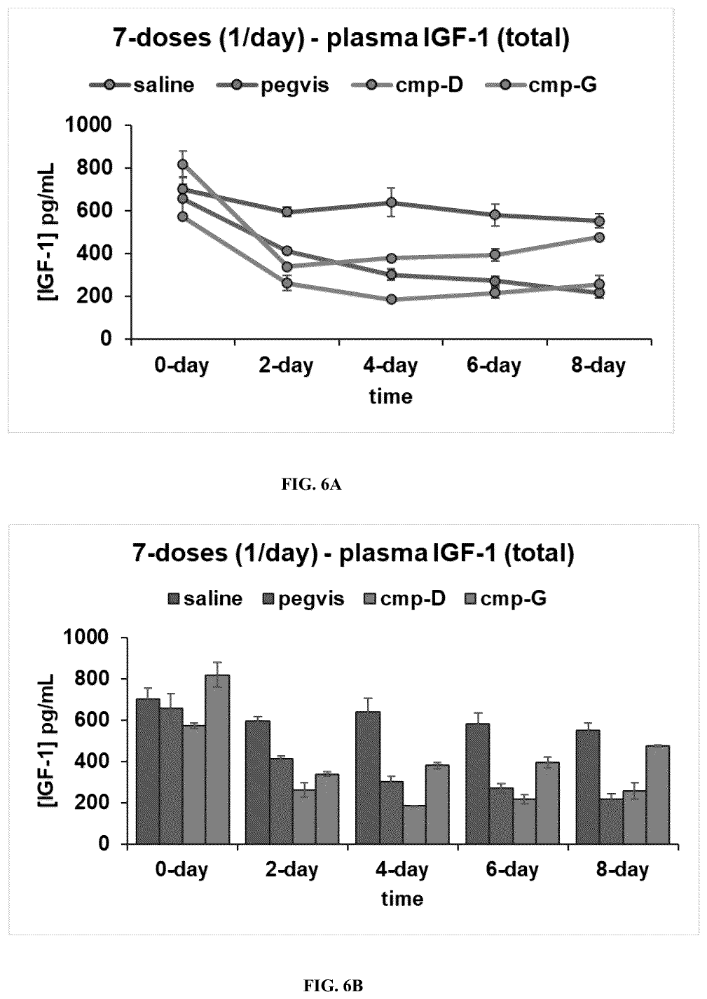

FIG. 3A and FIG. 3B provide data based on single intraperitoneal (IP) injections in an animal model demonstrating the efficacy of an alternate compound (Compound G disclosed in U.S. Pat. No. 11,452,763) in substantially reducing circulating IGF-1 levels. FIGS. 4A and 4B provide data based on daily IP injections for 7 days in an animal model demonstrating the efficacy of an alternate compound (Compound G disclosed in U.S. Pat. No. 11,452,763) in substantially reducing circulating IGF-1 levels. FIG. 5A and FIG. 5B provide data based on single intraperitoneal (IP) injections in an animal model demonstrating the efficacy of Compound D₂ compared to Compound G in substantially reducing circulating IGF-1 levels. FIG. 6A and FIG. 6B provide data based on daily IP injections for 7 days in an animal model demonstrating the efficacy of Compound D₂ compared to Compound G in substantially reducing circulating IGF-1 levels.

Advantages of the disclosed methodology include: (i) pleiotropic effect; (ii) a novel, next generation GHRA compound; and (iii) expedient translation for human use. Regarding pleiotropic effect, the etiology of AD is multifactorial. Prior attempts at therapeutically targeting individual pathogenic features have not been effective. However, Compound D₂, by way of attenuation of the GH/IGF-1 pathway, is expected to target most AD pathogenic features simultaneously, including improved insulin sensitivity and reduced neuroinflammation and, thus, exhibit a pleiotropic effect on AD. Pleiotropic targeting would significantly advance the approach to therapeutic development for AD. Regarding a novel, next generation GHRA compound, Compound D₂, has been engineered to have a slow clearance rate and greater affinity for the GHR compared to pegvisomant, making this molecule appealing and amenable for longer-term clinical use. Regarding expedient translation for human use, Compound D₂ is similar to a GHRA drug currently approved by the FDA (pegvisomant) for the treatment of acromegaly. Pegvisomant has been in clinical use since 2003 with excellent effect and safety record [22]. Compound D₂ is expected to be more efficacious but with the same known safety profile. Given its similarity to an approved therapeutic compound, if proven efficacious, Compound D₂ will, presumably, advance to human trials of AD expeditiously.

As previously indicated, the disclosed technology also provides a method for delaying aging and prolonging lifespan, comprising reducing circulating IGF-1 levels by administering to the patient an effective amount of a human growth hormone receptor antagonist that comprises human growth hormone receptor antagonist G120K, wherein a single amino acid of the human growth hormone receptor antagonist G120K has been mutated to cysteine, and wherein the single amino acid mutated to cysteine is T142; and a polydispersed 40 kDa branched polyethylene glycol molecule conjugated to the substituted cysteine in the human growth hormone receptor antagonist G120K mutant, wherein reducing circulating IGF-1 levels delays or slows aging and prolongs lifespan. This use of the disclosed human growth hormone receptor antagonist as an effective anti-aging compound based on its demonstrated reduction of circulating IGF-1 levels is supported by references cited below [51-64], all of which are incorporated by reference herein, for all purposes.

All literature and similar material cited in this application, including, but not limited to, patents, patent applications, articles, books, treatises, and web pages, regardless of the format of such literature and similar materials, are expressly incorporated by reference in their entirety. Should one or more of the incorporated references and similar materials differ from or contradict this application, including but not limited to defined terms, term usage, described techniques, or the like, this application controls.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about”, if or when used throughout this specification describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

There may be many alternate ways to implement the disclosed technology. Various functions and elements described herein may be partitioned differently from those shown without departing from the scope of the disclosed technology. Generic principles defined herein may be applied to other implementations. Different numbers of a given module or unit may be employed, a different type or types of a given module or unit may be employed, a given module or unit may be added, or a given module or unit may be omitted.

Regarding this disclosure, the term “a plurality of” refers to two or more than two. Unless otherwise clearly defined, orientation or positional relations indicated by terms such as “upper” and “lower” are based on the orientation or positional relations as shown in the Figures, only for facilitating description of the disclosed technology and simplifying the description, rather than indicating or implying that the referred devices or elements must be in a particular orientation or constructed or operated in the particular orientation, and therefore they should not be construed as limiting the disclosed technology. The terms “connected”, “mounted”, “fixed”, etc. should be understood in a broad sense. For example, “connected” may be a fixed connection, a detachable connection, or an integral connection, a direct connection, or an indirect connection through an intermediate medium. For one of ordinary skill in the art, the specific meaning of the above terms in the disclosed technology may be understood according to specific circumstances.

It should be appreciated that all combinations of the foregoing concepts and additional concepts discussed in greater detail herein (provided such concepts are not mutually inconsistent) are contemplated as being part of the disclosed technology. In particular, all combinations of claimed subject matter appearing at the end of this disclosure are contemplated as being part of the technology disclosed herein. While the disclosed technology has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed technology in its broader aspects is not limited to any of the specific details, representative devices and methods, and/or illustrative examples shown and described. Accordingly, departures may be made from such details without departing from the spirit or scope of the general inventive concept.

The following references form part of the specification of the present application and each reference is incorporated by reference herein, in its entirety, for all purposes.
1. Andersen S L, Sebastiani P, Dworkis D A, Feldman L, Perls T T. Health span approximates life span among many supercentenarians: compression of morbidity at the approximate limit of life span. J Gerontol A Biol Sci Med Sci. 2012; 67(4): 395-405.
2. Sebastiani P, Sun F X, Andersen S L, et al. Families Enriched for Exceptional Longevity also have Increased Health-Span: Findings from the Long Life Family Study. Front Public Health. 2013; 1:38.
3. Suh Y, Atzmon G, Cho M O, et al. Functionally significant insulin-like growth factor I receptor mutations in centenarians. Proc Natl Acad Sci USA. 2008; 105(9): 3438-3442.
4. Tazearslan C, Huang J, Barzilai N, Suh Y. Impaired IGFIR signaling in cells expressing longevity-associated human IGFIR alleles. Aging Cell. 2011; 10(3): 551-554.
5. Brown-Borg H M, Borg K E, Meliska C J, Bartke A. Dwarf mice and the ageing process. Nature. 1996; 384(6604): 33.
6. Ikeno Y, Bronson R T, Hubbard G B, Lee S, Bartke A. Delayed occurrence of fatal neoplastic diseases in ames dwarf mice: correlation to extended longevity. J Gerontol A Biol Sci Med Sci. 2003; 58(4): 291-296.
7. Kenyon C, Chang J, Gensch E, Rudner A, Tabtiang R. A C. elegans mutant that lives twice as long as wild type. Nature. 1993; 366(6454): 461-464.
8. Kinney B A, Coschigano K T, Kopchick J J, Steger R W, Bartke A. Evidence that age-induced decline in memory retention is delayed in growth hormone resistant GH-R-KO (Laron) mice. Physiology & behavior. 2001; 72(5): 653-660.
9. Kinney B A, Meliska C J, Steger R W, Bartke A. Evidence that Ames dwarf mice age differently from their normal siblings in behavioral and learning and memory parameters. Hormones and behavior. 2001; 39(4): 277-284.
10. Ben-Avraham D, Govindaraju D R, Budagov T, et al. The GH receptor exon 3 deletion is a marker of male-specific exceptional longevity associated with increased GH sensitivity and taller stature. Science advances. 2017; 3(6):e1602025.
11. Levine M E, Suarez J A, Brandhorst S, et al. Low protein intake is associated with a major reduction in IGF-1, cancer, and overall mortality in the 65 and younger but not older population. Cell Metab. 2014; 19(3): 407-417.
12. Milman S, Atzmon G, Huffman D M, et al. Low insulin-like growth factor-1 level predicts survival in humans with exceptional longevity. Aging Cell. 2014.
13. Perice L, Barzilai N, Verghese J, et al. Lower circulating insulin-like growth factor-I is associated with better cognition in females with exceptional longevity without compromise to muscle mass and function. Aging (Albany NY). 2016; 8(10): 2414-2424.
14. van der Spoel E, Rozing M P, Houwing-Duistermaat J J, et al. Association analysis of insulin-like growth factor-1 axis parameters with survival and functional status in nonagenarians of the Leiden Longevity Study. Aging (Albany NY). 2015; 7(11): 956-963.
15. Zhang W B, Aleksic S, Gao T, et al. Insulin-like Growth Factor-1 and IGF Binding Proteins Predict All-Cause Mortality and Morbidity in Older Adults. Cells. 2020; 9(6).
16. Zhang W B, Ye K, Barzilai N, Milman S. The antagonistic pleiotropy of insulin-like growth factor 1. Aging Cell. 2021; 20(9):e13443.
17. Hong S, Han K, Kim K S, Park C Y. The Risk of Neurodegenerative Diseases in Patients With Acromegaly: A Cohort Study. Neurology. 2022.
18. Cohen E, Paulsson J F, Blinder P, et al. Reduced IGF-1 signaling delays age-associated proteotoxicity in mice. Cell. 2009; 139(6): 1157-1169.
19. Gontier G, George C, Chaker Z, Holzenberger M, Aid S. Blocking IGF Signaling in Adult Neurons Alleviates Alzheimer's Disease Pathology through Amyloid-beta Clearance. The Journal of neuroscience: the official journal of the Society for Neuroscience. 2015; 35(33): 11500-11513.
20. Puig K L, Kulas J A, Franklin W, et al. The Ames dwarf mutation attenuates Alzheimer's disease phenotype of APP/PS1 mice. Neurobiology of aging. 2016; 40:22-40.
21. Basu A, McFarlane H G, Kopchick J J. Spatial learning and memory in male mice with altered growth hormone action. Hormones and behavior. 2017; 93:18-30.
22. Fleseriu M, Fuhrer-Sakel D, van der Lely A J, et al. More than a decade of real-world experience of pegvisomant for acromegaly: ACROSTUDY. Eur J Endocrinol. 2021; 185(4): 525-538.
23. van Heemst D, Beekman M, Mooijaart S P, et al. Reduced insulin/IGF-1 signalling and human longevity. Aging Cell. 2005; 4(2): 79-85.
24. Silver M H, Jilinskaia E, Perls T T. Cognitive functional status of age-confirmed centenarians in a population-based study. The journals of gerontology Series B, Psychological sciences and social sciences. 2001; 56(3): P134-140.
25. Ohlsson C, Mohan S, Sjogren K, et al. The role of liver-derived insulin-like growth factor-I. Endocr Rev. 2009; 30(5): 494-535.
26. Carro E, Spuch C, Trejo J L, Antequera D, Torres-Aleman I. Choroid plexus megalin is involved in neuroprotection by serum insulin-like growth factor I. The Journal of neuroscience: the official journal of the Society for Neuroscience. 2005; 25(47): 10884-10893.
27. Holly J M, Perks C M. Insulin-like growth factor physiology: what we have learned from human studies. Endocrinol Metab Clin North Am. 2012; 41(2): 249-263, v.
28. Baxter R C. Insulin-like growth factor (IGF)-binding proteins: interactions with IGFs and intrinsic bioactivities. Am J Physiol Endocrinol Metab. 2000; 278(6):E967-976.
29. Rajaram S, Baylink D J, Mohan S. Insulin-like growth factor-binding proteins in serum and other biological fluids: regulation and functions. Endocr Rev. 1997; 18(6): 801-831.
30. Taniguchi C M, Emanuelli B, Kahn C R. Critical nodes in signalling pathways: insights into insulin action. Nature reviews Molecular cell biology. 2006; 7(2): 85-96.
31. Roczniak-Ferguson A, Petit C S, Froehlich F, et al. The transcription factor TFEB links mTORC1 signaling to transcriptional control of lysosome homeostasis. Science signaling. 2012; 5(228): ra42.
32. Martinez-Vicente M, Cuervo A M. Autophagy and neurodegeneration: when the cleaning crew goes on strike. The Lancet Neurology. 2007; 6(4): 352-361.
33. Yamamoto H, Sohmiya M, Oka N, Kato Y. Effects of aging and sex on plasma insulin-like growth factor I (IGF-I) levels in normal adults. Acta Endocrinol (Copenh). 1991; 124(5): 497-500.
34. Carlson H E, Gillin J C, Gorden P, Snyder F. Abscence of sleep-related growth hormone peaks in aged normal subjects and in acromegaly. J Clin Endocrinol Metab. 1972; 34(6): 1102-1105.
35. Finkelstein J W, Roffwarg H P, Boyar R M, Kream J, Hellman L. Age-related change in the twenty-four-hour spontaneous secretion of growth hormone. J Clin Endocrinol Metab. 1972; 35(5): 665-670.
36. Abuzzahab M J, Schneider A, Goddard A, et al. IGF-I receptor mutations resulting in intrauterine and postnatal growth retardation. N Engl J Med. 2003; 349(23): 2211-2222.
37. Kranzler J H, Rosenbloom A L, Martinez V, Guevara-Aguirre J. Normal intelligence with severe insulin-like growth factor I deficiency due to growth hormone receptor deficiency: a controlled study in a genetically homogeneous population. J Clin Endocrinol Metab. 1998; 83(6): 1953-1958.
38. Netchine I, Azzi S, Le Bouc Y, Savage M O. IGF1 molecular anomalies demonstrate its critical role in fetal, postnatal growth and brain development. Best practice & research Clinical endocrinology & metabolism. 2011; 25(1): 181-190.
39. Woods K A, Camacho-Hubner C, Savage M O, Clark A J. Intrauterine growth retardation and postnatal growth failure associated with deletion of the insulin-like growth factor I gene. N Engl J Med. 1996; 335(18): 1363-1367.
40. Bianchi V E, Locatelli V, Rizzi L. Neurotrophic and Neuroregenerative Effects of GH/IGF1. International journal of molecular sciences. 2017; 18(11).
41. Friedlander A L, Butterfield G E, Moynihan S, et al. One year of insulin-like growth factor I treatment does not affect bone density, body composition, or psychological measures in postmenopausal women. J Clin Endocrinol Metab. 2001; 86(4): 1496-1503.
42. Sevigny J J, Ryan J M, van Dyck C H, et al. Growth hormone secretagogue MK-677: no clinical effect on AD progression in a randomized trial. Neurology. 2008; 71(21): 1702-1708.
43. Nashiro K, Guevara-Aguirre J, Braskie M N, et al. Brain Structure and Function Associated with Younger Adults in Growth Hormone Receptor-Deficient Humans. The Journal of neuroscience: the official journal of the Society for Neuroscience. 2017; 37(7): 1696-1707.
44. Freude S, Hettich M M, Schumann C, et al. Neuronal IGF-1 resistance reduces Abeta accumulation and protects against premature death in a model of Alzheimer's disease. Faseb J. 2009; 23(10): 3315-3324.
45. Jaszberenyi M, Rick F G, Szalontay L, et al. Beneficial effects of novel antagonists of GHRH in different models of Alzheimer's disease. Aging (Albany NY). 2012; 4(11): 755-767.
46. Bitto A, Lerner C, Torres C, et al. Long-term IGF-I exposure decreases autophagy and cell viability. PLOS One. 2010; 5(9):e12592.
47. George C, Gontier G, Lacube P, Francois J C, Holzenberger M, Aid S. The Alzheimer's disease transcriptome mimics the neuroprotective signature of IGF-1 receptor-deficient neurons. Brain: a journal of neurology. 2017; 140(7): 2012-2027.
48. Talbot K, Wang H Y, Kazi H, et al. Demonstrated brain insulin resistance in Alzheimer's disease patients is associated with IGF-1 resistance, IRS-1 dysregulation, and cognitive decline. J Clin Invest. 2012; 122(4): 1316-1338.
49. Muller A P, Fernandez A M, Haas C, Zimmer E, Portela L V, Torres-Aleman I. Reduced brain insulin-like growth factor I function during aging. Molecular and cellular neurosciences. 2012; 49(1): 9-12.
50. Trueba-Saiz A, Cavada C, Fernandez A M, et al. Loss of serum IGF-I input to the brain as an early biomarker of disease onset in Alzheimer mice. Translational psychiatry. 2013; 3:e330.
51. Zhang W B, Ye K, Barzilai N, Milman S. The antagonistic pleiotropy of insulin-like growth factor 1. Aging Cell 2021; 20(9), e13443; PMCID: PMC8441393.
52. DeVito L M, Barzilai N, Cuervo A M, Niedernhofer L J, Milman S, Levine M, Promislow D, Ferrucci L, Kuchel G A, Mannick J, Justice J, Gonzales M M, Kirkland J L, Cohen P, Campisi J. Extending human healthspan and longevity: a symposium report. Ann NY Acad Sci 2021.
53. Zhang W B, Aleksic S, Gao T, Weiss E F, Demetriou E, Verghese J, Holtzer R, Barzilai N, Milman S. Insulin-like Growth Factor-1 and IGF Binding Proteins Predict All-Cause Mortality and Morbidity in Older Adults. Cells 2020; 9(6):E1368, PMCID: PMC7349399.
54. Zhang Z D, Milman S, Lin J, Barzilai N, Gorbunova V, Ladiges W C, Niedernhofer L F, Suh Y, Robbins P D, Vijg J. Genetics of extreme human longevity to guide drug discovery for healthy ageing. Nat Metab 2020; 2(8) 663-672.
55. Lehallier B, Gate D, Schaum N, Nanasi T, Lee S E, Yousef H, Moran Losada P, Berdnik D, Keller A, Verghese J, Sathyan S, Franceschi C, Milman S, Barzilai N, Wyss-Coray T. Undulating changes in human plasma proteome profiles across the lifespan. Nat Med 2019; 25(12): 1843-1850; PMCID: PMC7062043.
56. Gubbi S, Quipildor G F, Barzilai N, Huffman D M, Milman S. 40 Years of IGF-1: IGF-1: the Jekyll and Hyde of the aging brain. J Mol Endocrinol 2018; 61(1): T171-T185. PMCID: PMC5988994.
57. Gubbi S, Schwartz E, Crandall J P, Verghese J, Holtzer R, Atzmon G, Braunstein R, Barzilai N, Milman S. Effect of Exceptional Parental Longevity and Lifestyle Factors on Prevalence of Cardiovascular Disease in Offspring. Am J Cardiol 2017.
58. Ismail K, Nussbaum L, Sebastiani P, Andersen S, Perls T, Barzilai N, Milman S. Compression of Morbidity is Observed Across Cohorts with Exceptional Longevity. J Am Geriatr Soc 2016; 64(8): 1583-91. PMCID: PMC4988893.
59. Milman S, Huffman D M, Barzilai N. The Somatotropic Axis in Human Aging: Framework for the Current State of Knowledge and Future Research. Cell Metab 2016; 23(6): 980-9. PMCID: PMC4919980.
60. Perice L, Barzilai N, Verghese J, Weiss E F, Holtzer R, Cohen P, Milman S. Lower Circulating Insulin-like Growth Factor-I is Associated with Better Cognition in Females with Exceptional Longevity Without Compromise to Muscle Mass and Function. Aging Albany NY) 2016; 8(10): 2414-2424. PMCID: PMC5115897.
61. Milman S, Barzilai N. Dissecting the Mechanisms Underlying Unusually Successful Human Health Span and Life Span. Cold Spring Harb Perspect Med 2015 Dec. 4. PMCID: PMC4691799.
62. Milman S, Atzmon G, Huffman D M, Wan J, Crandall J P, Cohen P, Barzilai N. Low Insulin-like Growth Factor-1 Level Predicts Survival in Humans with Exceptional Longevity. Aging Cell 2014; 13(4): 769-71.
63. Kopchick J, Berryman D, List E. Chasing Methuselah: adult inducible GHRKO mice. Aging (Albany NY). 2022 Apr. 30; 14(8): 3331-3332.
64. Nashiro K, Guevara-Aguirre J, Braskie M, Hafzalla G, Velasco R, Balasubramanian P, Wei M, Thompson P, Mather M, Nelson M, Guevara A, Teran E, Longo V. Brain Structure and Function Associated with Younger Adults in Growth Hormone Receptor-Deficient Humans. Journal of Neuroscience 15 Feb. 2017, 37(7) 1696-1707.

What is claimed:

1. A method for treating diseases or conditions responsive to human growth hormone receptor antagonists, comprising administering to the patient an effective amount of a human growth hormone receptor antagonist, comprising:
(a) human growth hormone receptor antagonist G120K, wherein a single amino acid of the human growth hormone receptor antagonist G120K has been mutated to cysteine, and wherein the single amino acid mutated to cysteine is T142; and
(b) a polydispersed 40 kDa branched polyethylene glycol molecule conjugated to the substituted cysteine in the human growth hormone receptor antagonist G120K mutant.

2. The method of claim 1, wherein the diseases or conditions include Alzheimer's disease and related dementias.

3. The method of claim 1, wherein the diseases or conditions include aging.

4. The method of claim 1, wherein administering the human growth hormone receptor antagonist reduces circulating IGF-1 levels.

5. The method of claim 1, wherein the human growth hormone receptor antagonist is encoded by a DNA molecule having the sequence consisting of SEQ ID NO: 7, and wherein the human growth hormone receptor antagonist has the amino acid sequence consisting of SEQ ID NO: 8.

6. The method of claim 1, wherein the human growth hormone receptor antagonist is encoded by a DNA molecule having the sequence consisting of SEQ ID NO: 9, and wherein the human growth hormone receptor antagonist has the amino acid sequence consisting of SEQ ID NO: 10.

7. The method of claim 1, wherein the human growth hormone receptor antagonist is administered to a mammal.

8. A method for treating Alzheimer's disease and related dementias, comprising:
(a) reducing circulating IGF-1 levels by
(b) administering to the patient an effective amount of a human growth hormone receptor antagonist, comprising:
(i) human growth hormone receptor antagonist G120K, wherein a single amino acid of the human growth hormone receptor antagonist G120K has been mutated to cysteine, and wherein the single amino acid mutated to cysteine is T142; and
(ii) a polydispersed 40 kDa branched polyethylene glycol molecule conjugated to the substituted cysteine in the human growth hormone receptor antagonist G120K mutant, wherein
(c) reducing circulating IGF-1 levels reduces the effects of Alzheimer's disease and related dementias.

9. The method of claim 8, wherein the human growth hormone receptor antagonist is encoded by a DNA molecule having the sequence consisting of SEQ ID NO: 7, and wherein the human growth hormone receptor antagonist has the amino acid sequence consisting of SEQ ID NO: 8.

10. The method of claim 8, wherein the human growth hormone receptor antagonist is encoded by a DNA molecule having the sequence consisting of SEQ ID NO: 9, and wherein the human growth hormone receptor antagonist has the amino acid sequence consisting of SEQ ID NO: 10.

11. The method of claim 8, wherein the human growth hormone receptor antagonist is administered to a mammal.

12. A method of delaying aging and prolonging lifespan, comprising,
(a) reducing circulating IGF-1 levels by
(b) administering to the patient an effective amount of a human growth hormone receptor antagonist, comprising:
(i) human growth hormone receptor antagonist G120K, wherein a single amino acid of the human growth hormone receptor antagonist G120K has been mutated to cysteine, and wherein the single amino acid mutated to cysteine is T142; and
(ii) a polydispersed 40 kDa branched polyethylene glycol molecule conjugated to the substituted cysteine in the human growth hormone receptor antagonist G120K mutant, wherein
(c) reducing circulating IGF-1 levels delays or slows aging and prolongs lifespan.

13. The method of claim 12, wherein the human growth hormone receptor antagonist is encoded by a DNA molecule having the sequence consisting of SEQ ID NO: 7, and wherein the human growth hormone receptor antagonist has the amino acid sequence consisting of SEQ ID NO: 8.

14. The method of claim 12, wherein the human growth hormone receptor antagonist is encoded by a DNA molecule having the sequence consisting of SEQ ID NO: 9, and wherein the human growth hormone receptor antagonist has the amino acid sequence consisting of SEQ ID NO: 10.

15. The method of claim 12, wherein the human growth hormone receptor antagonist is administered to a mammal.
